(19) United States
(12) Patent Application Publication
(10) Pub. No.: US 2004/0258772 A1
(43) Pub. Date: Dec. 23, 2004

(54) Methods of treating angiogenesis, tumor growth, and metastasis

(76) Inventors: Leo E. Otterbein, New Kensington, PA (US); Augustine M. K. Choi, Pittsburgh, PA (US)

Correspondence Address: FISH & RICHARDSON PC, 225 FRANKLIN ST, BOSTON, MA, 02110, US

(21) Appl. No.: 10/455,564

(22) Filed: Jun. 5, 2003

Related U.S. Application Data
(60) Provisional application No. 60/386,561, filed on Jun. 5, 2002.

(51) Int. Cl. A61K 33/00

(52) U.S. Cl. 424/699

(57) ABSTRACT

The present invention relates to a method of treating cancer or unwanted angiogenesis in a patient, which includes administering a pharmaceutical composition that includes carbon monoxide to the patient.

### CROSS-REFERENCE TO RELATED APPLICATIONS

This application claims priority to U.S. Provisional Application No. 60/386,561 filed Jun. 5, 2002, which is incorporated herein by reference in its entirety.

### STATEMENT AS TO FEDERALLY SPONSORED RESEARCH

This invention was made with Government support under National Institutes of Health Grant Nos. HL55330, HL 60234 and AI 42365. The Government has certain rights in this invention.

### TECHNICAL FIELD

This invention generally relates to the treatment of cancer and angiogenesis.

### BACKGROUND

Carbon monoxide gas is poisonous in high concentrations. However, it is now recognized as an important signaling molecule (Venna et al., Science 259:381-384, 1993). It has also been suggested that carbon monoxide acts as a neuronal messenger molecule in the brain (Id.) and as a neuro-endocrine modulator in the hypothalamus (Pozzoli et al., Endocrinology 735:2314-2317, 1994). Like nitric oxide (NO), carbon monoxide is a smooth muscle relaxant (Utz et al., Biochem Pharmacol. 47:195-201, 1991; Christodoulides et al., Circulation 97:2306-9, 1995) and inhibits platelet aggregation (Mansouri et al., Thromb Haemost. 48:286-8, 1982). Inhalation of low levels of carbon monoxide (CO) has been shown to have anti-inflammatory effects in some models.

Cancer is a disease characterized by a proliferation of cells that have malfunctioning cellular regulatory systems. The malfunctioning cell regulatory systems can result in unregulated growth of the cells, lack of cellular differentiation, local tissue invasion by the cells, and metastasis. The treatment of existing tumors and disseminated cancer cells (metastases) is a fundamental problem in clinical medicine.

Angiogenesis is the formation of new capillary blood vessels, and is an important component in pathologic processes such as chronic inflammation, certain immune responses, and cancer. Angiogenesis is also involved in normal processes such as embryo development and wound healing.

### SUMMARY

The present invention is based, in part, on the discovery that administration of CO can inhibit the growth of tumor cells in vitro and whole tumors in vivo. Furthermore, it has now been found that administration of CO can suppress angiogenesis. The present invention provides, for example, methods of treating tumors and metastases using pharmaceutical compositions comprising CO.

Accordingly, the present invention features a method of treating cancer, preventing cancer, or reducing the risk of cancer, e.g., naturally arising cancer, in a patient. The method includes administering to (and/or prescribing for) a patient identified (e.g., diagnosed) as suffering from (or at elevated risk for) cancer a therapeutically effective amount of a composition comprising carbon monoxide.

The pharmaceutical composition used in this or any of the other treatment methods described below can be in gaseous or liquid form, and can be administered to the patient by any method known in the art for administering gases and liquids to patients, e.g., via inhalation, insufflation, infusion, injection, and/or ingestion. In one embodiment of the present invention, the pharmaceutical composition is in gaseous or liquid (e.g., in the form of a mist or spray) form, and is administered to the patient by inhalation. If in liquid form, the pharmaceutical composition can also be administered to the patient orally. In another embodiment, the pharmaceutical composition is in gaseous and/or liquid form, and is administered topically to an organ of the patient. In yet another embodiment, the pharmaceutical composition is in gaseous and/or liquid form, and is administered directly to the abdominal cavity of the patient. The pharmaceutical composition can also be administered to the patient by an extracorporeal membrane gas exchange device or an artificial lung.

The methods can be used alone or in combination with other methods for treating cancer in patients. Accordingly, in another embodiment, the methods described herein can include treating the patient using surgery (e.g., to remove a tumor, or portion thereof), chemotherapy, immunotherapy, gene therapy, and/or radiation therapy. A pharmaceutical composition comprising carbon monoxide as described herein can be administered to a patient at any point, e.g., before, during, and/or after the surgery, chemotherapy, immunotherapy, gene therapy, and/or radiation therapy.

The patient is an animal, human or non-human, and rodent or non-rodent. For example, the patient can be any mammal, e.g., a human, other primate, pig, rodent such as mouse or rat, rabbit, guinea pig, hamster, cow, horse, cat, dog, sheep or goat, or a non-mammal such as a bird. The cancer can be the result of any of a number of factors, e.g., carcinogens; infections, e.g., viral infections; radiation; and/or heredity, or can be of indeterminate origin. The pharmaceutical composition can be in any form, e.g., gaseous or liquid form.

Methods described herein can be carried out along with at least one of the following treatments: inducing HO-1 or ferritin in the patient; expressing HO-1 or ferritin in the patient; and administering a pharmaceutical composition comprising HO-1, bilirubin, biliverdin, ferritin, iron, desferoxamine, iron dextran and/or apoferritin to the patient.

Also included in the present invention is a method of treating cancer in a patient, which includes determining whether cancerous cells in a patient express p21, and administering to the patient a therapeutically effective amount of a composition comprising carbon monoxide if the cancerous cells express p21. The method can optionally include a step of identifying (e.g., diagnosing) the patient as suffering from cancer.

The present invention also includes a method of performing chemotherapy, immunotherapy, gene therapy, and/or radiation therapy on a patient. The method includes administering chemotherapy, immunotherapy, gene therapy, and/or radiation therapy to the patient, and administering to the patient a therapeutically effective amount of a composition comprising carbon monoxide. The composition can be administered at any time in the method, e.g., before and/or during and/or after the administration of chemotherapy, immunotherapy, gene therapy, and/or radiation therapy to the patient. The method can optionally include a step of identifying (e.g., diagnosing) a patient as being in need of chemotherapy, radiation therapy immunotherapy, and/or gene therapy.

Also included in the present invention is a method of performing surgery to remove cancer, e.g., naturally arising cancer, from a patient. The method includes identifying a patient in need of surgery to remove cancer from the patient and/or identifying at least one cancerous tissue-bearing organ in a patient, performing surgery on the patient to remove cancerous tissue, and administering to the patient (either systemically (e.g., by inhalation) or locally at the site of surgery) a therapeutically effective amount of a composition comprising carbon monoxide. The composition can be administered at any time in the procedure, e.g., before and/or during and/or after performing surgery on the patient.

In another aspect, the invention features a method of treating or preventing (i.e., reducing the risk of) cancer in a patient, which includes identifying a patient suffering from or at risk for a cancer, providing a vessel containing a pressurized gas comprising carbon monoxide gas, releasing the pressurized gas from the vessel to form an atmosphere comprising carbon monoxide gas, and exposing the patient to the atmosphere, wherein the amount of carbon monoxide in the atmosphere is sufficient to treat or reduce the risk of cancer.

The patient can be exposed to the pharmaceutical composition or CO-containing atmosphere over any period of time, including indefinitely. Preferred periods of time include at least one hour, e.g., at least six hours; at least one day; at least one week, two weeks, four weeks, six weeks, eight weeks, ten weeks or twelve weeks; at least one year; at least two years; and at least five years. The patient can be exposed to the atmosphere continuously or intermittently during such periods.

In methods described herein, the cancer can be cancer found in any part(s) of the patent's body, e.g., cancer of the stomach, small intestine, colon, rectum, mouth/pharynx, esophagus, larynx, liver, pancreas, lung, breast, cervix uteri, corpus uteri, ovary, prostate, testis, bladder, skin, kidney, brain/central nervous system, head, neck, throat, or any combination thereof.

The concentration of carbon monoxide in the inhaled gas can be any concentration described herein, e.g., about 0.0001% to about 0.25% by weight. In preferred embodiments, the concentration of carbon monoxide in the inhaled gas is about 0.005% to about 0.24%, or about 0.01% to about 0.22% by weight. More preferably, the concentration of carbon monoxide in the inhaled gas is about 0.025% to about 0.1% by weight.

In another aspect, the invention features a method of treating unwanted angiogenesis in a patient. The method includes administering to a patient diagnosed as suffering from or at risk for unwanted angiogenesis a therapeutically effective amount of a composition comprising carbon monoxide. The method can optionally include a step of identifying (e.g., diagnosing) the patient as suffering from or at risk for unwanted angiogenesis.

The composition can be in gaseous form and administered to the patient via inhalation, topically to an organ of the patient and/or to the abdominal cavity of the patient. In another embodiment, the composition can be in liquid form and administered to the patient orally, topically to an organ of the patient, and/or to the abdominal cavity of the patient.

In still another aspect, the invention features a method of treating a condition associated with unwanted angiogenesis. The method includes administering to a patient diagnosed as suffering from or at risk for a condition associated with unwanted angiogenesis a therapeutically effective amount of a composition comprising carbon monoxide, wherein the condition associated with unwanted angiogenesis is not cancer. The method can optionally include a step of identifying (e.g., diagnosing) the patient as suffering from or at risk for a condition associated with unwanted angiogenesis. In an embodiment, the condition is rheumatoid arthritis, lupus, psoriasis, diabetic retinopathy, retinopathy of prematurity, macular degeneration, corneal graft rejection, neovascular glaucoma, retrolental fibroplasia, rubeosis, Osler-Weber Syndrome, myocardial angiogenesis, plaque neovascularization, telangiectasia, or angiofibroma, or any combination thereof.

In another aspect, the invention provides a vessel comprising medical grade compressed CO gas. The vessel can bear a label indicating that the gas can be used to treat cancer in a patient. Alternatively or in addition, the vessel can bear a label indicating that the gas can be administered to a patient to treat (e.g., prevent or reduce) unwanted angiogenesis, or a condition associated with unwanted angiogenesis, in the patient. The CO gas can be supplied as an admixture with nitrogen gas, with nitric oxide and nitrogen gas, or with an oxygen-containing gas. The CO gas can be present in the admixture at a concentration of at least about 0.025%, e.g., at least about 0.05%, 0.10%, 0.50%, 1.0%, 2.0%, 10%, 50%, or 90%.

Also within the invention is the use of CO in the manufacture of a medicament for treatment or prevention of a condition described herein, e.g., cancer, unwanted angiogenesis, and/or a condition (e.g., other than cancer) associated with unwanted angiogenesis. The medicament can be used in a method for treating cancer, for preventing angiogenesis, and/or for treating a condition associated with unwanted angiogenesis in accordance with the methods described herein. The medicament can be in any form described herein, e.g., a liquid or gaseous CO composition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. The materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 is a line graph illustrating that CO inhibits the proliferation of mouse mesothelioma (AC29) cells. Closed circles represent cells exposed to air. Closed squares represent cells exposed to CO. The arrow indicates a time point at which cells were removed from the CO-containing environment.

FIG. 2 is a bar graph illustrating that human adenocarcinoma (A549) cells that have been transfected with the HO-1 gene (which causes the cells to overexpress HO-1 protein) exhibit reduced tumor volume in mice. Wt=Wild type A549 cells (control); NEO=A549 cells transfected with vector alone (control); HO-1 Clones A5 and L1=two distinct lines of A549 cells transfected with the HO-1 gene.

FIG. 3 is a line graph illustrating that exposure to CO prolongs survival in mice injected with a lethal number of mesothelioma cells. Closed circles represent mice exposed to air. Closed squares represent mice exposed to CO. The arrow indicates a time point at which half of the CO-exposed mice were removed from the CO chamber.

FIG. 4 is a line graph illustrating that exposure to CO prolongs survival in mice injected with a lethal number of mesothelioma cells when CO exposures begin one week after the injections. Closed circles represent mice exposed to air. Closed squares represent mice exposed to CO.

FIG. 5 is a bar graph illustrating that CO-induced growth arrest in A549 cells is cGMP dependent. Cells were exposed in vitro to: air; CO; CO+1H-[1,2,4] Oxadiazolo [4,3-a]quinoxalin-1-one (ODQ); or CO+Rp-8-Bromo-cGMP (Rp8-Br).

FIG. 6 is a bar graph illustrating that CO-induced growth arrest is less marked in human colon cancer cells (HTC) that are deficient in p21. Wt=wild type HTC cells; WT+CO=wild type cells exposed to CO; p21−/−=HTC cells deficient in p21; p21−/−+CO=HTC cells deficient in p21 exposed to CO.

FIG. 7 is a bar graph illustrating that CO inhibits vascular endothelial growth factor (VEGF) production by A549 cells. Air=A549 cells exposed to air; CO=A549 cells exposed to CO.

FIG. 8 is a bar graph illustrating that tumor volume is reduced in mice injected with A549 cells and exposed to CO plus air (CO) as compared to mice injected with A549 cells and exposed to air alone (Air).

FIG. 9A is a composite picture of immunoblots illustrating that exposure of A549 cells to CO over a 24 hour period causes changes in expression of p21, p27, proliferating cell nuclear antigen (PCNA), Cdc25b, and cyclin D1. Lane 1=cells exposed to CO for 0 hrs; Lane 2=cells exposed to CO for 24 hrs.

FIG. 9B is a picture of an immunoblot illustrating that exposure of A549 cells to CO over periods of 4, 8, and 24 hours causes changes in expression of p21.

### DETAILED DESCRIPTION

The term “carbon monoxide” (or “CO”) as used herein describes molecular carbon monoxide in its gaseous state, compressed into liquid form, or dissolved in aqueous solution. The terms “carbon monoxide composition” and “pharmaceutical composition comprising carbon monoxide” are used throughout the specification to describe a gaseous or liquid composition containing carbon monoxide that can be administered to a patient and/or an organ, e.g., an organ affected by cancer. The skilled practitioner will recognize which form of the pharmaceutical composition, e.g., gaseous, liquid, or both gaseous and liquid forms, is preferred for a given application.

The terms “effective amount” and “effective to treat,” as used herein, refer to an amount or concentration of carbon monoxide utilized for a period of time (including acute or chronic administration and periodic or continuous administration) that is effective within the context of its administration for causing an intended effect or physiological outcome. Effective amounts of carbon monoxide for use in the present invention include, for example, amounts that inhibit the growth of cancer, e.g., tumors and/or tumor cells, improve the outcome for a patient suffering from or at risk for cancer, and improve the outcome of other cancer treatments.

Effective amounts of carbon monoxide also include, for example, amounts that advantageously affect angiogenesis, production of vascular endothelial growth factor, and/or any of the cellular mechanisms involved in the inhibition of tumor growth described herein.

For gases, effective amounts of carbon monoxide in a composition generally fall within the range of about 0.0000001% to about 0.3% by weight, e.g., 0.0001% to about 0.25% by weight, preferably at least about 0.001%, e.g., at least 0.005%, 0.010%, 0.02%, 0.025%, 0.03%, 0.04%, 0.05%, 0.06%, 0.08%, 0.10%, 0.15%, 0.20%, 0.22%, or 0.24% by weight carbon monoxide. Preferred ranges include, e.g., 0.001% to about 0.24%, about 0.005% to about 0.22%, about 0.005% to about 0.05%, about 0.010% to about 0.20%, about 0.02% to about 0.15%, about 0.025% to about 0.10%, or about 0.03% to about 0.08%, or about 0.04% to about 0.06%. For liquid solutions of CO, effective amounts generally fall within the range of about 0.0001 to about 0.0044 g CO/100 g liquid, e.g., at least 0.0001, 0.0002, 0.0004, 0.0006, 0.0008, 0.0010, 0.0013, 0.0014, 0.0015, 0.0016, 0.0018, 0.0020, 0.0021, 0.0022, 0.0024, 0.0026, 0.0028, 0.0030, 0.0032, 0.0035, 0.0037, 0.0040, or 0.0042 g CO/100 g aqueous solution. Preferred ranges include, e.g., about 0.0010 to about 0.0030 g CO/100 g liquid, about 0.0015 to about 0.0026 g CO/100 g liquid, or about 0.0018 to about 0.0024 g CO/100 g liquid. A skilled practitioner will appreciate that amounts outside of these ranges may be used, depending upon the application.

The term “patient” is used throughout the specification to describe an animal, human or non-human, to whom treatment according to the methods of the present invention is provided. Veterinary applications are clearly anticipated by the present invention. The term includes but is not limited to birds, reptiles, amphibians, and mammals, e.g., humans, other primates, pigs, rodents such as mice and rats, rabbits, guinea pigs, hamsters, cows, horses, cats, dogs, sheep and goats. Preferred subjects are humans, farm animals, and domestic pets such as cats and dogs. The term “treat(ment),” is used herein to denote delaying the onset of, inhibiting, alleviating the effects of, or prolonging the life of a patient suffering from, a condition, e.g., cancer.

Examples of cellular proliferative and/or differentiative disorders include cancer, e.g., carcinoma, sarcoma, metastatic disorders and hematopoietic neoplastic disorders, e.g., leukemias.

A metastatic tumor can arise from a multitude of primary tumor types, including but not limited to those of prostate, colon, lung, breast, bone, and liver origin. Metastases develop, e.g., when tumor cells shed from a primary tumor adhere to vascular endothelium, penetrate into surrounding tissues, and grow to form independent tumors at sites separate from a primary tumor.

The term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

Cancers that may be treated using the methods and compositions of the present invention include, for example, cancers of the stomach, colon, rectum, mouth/pharynx, esophagus, larynx, liver, pancreas, lung, breast, cervix uteri, corpus uteri, ovary, prostate, testis, bladder, skin, bone, kidney, brain/central nervous system, head, neck and throat; Hodgkins disease, non-Hodgkins leukemia, sarcomas, choriocarcinoma, and lymphoma, among others.

Individuals considered at risk for developing cancer may benefit particularly from the invention, primarily because prophylactic treatment can begin before there is any evidence of the disorder. Individuals “at risk” include, e.g., individuals exposed to carcinogens, e.g., by consumption, e.g., by inhalation and/or ingestion, at levels that have been shown statistically to promote cancer in susceptable individuals. Also included are individuals at risk due to exposure to ultraviolet radiation, or their environment, occupation, and/or heredity, as well as those who show signs of a precancerous condition such as polyps. Similarly, individuals in very early stages of cancer or development of metastases (i.e., only one or a few aberrant cells are present in the individual's body or at a particular site in an individual's tissue)) may benefit from such prophylactic treatment.

Skilled practitioners will appreciate that a patient can be diagnosed by a physician (or veterinarian, as appropriate for the patient being diagnosed) as suffering from or at risk for a condition described herein, e.g., cancer, by any method known in the art, e.g., by assessing a patient's medical history, performing diagnostic tests, and/or by employing imaging techniques. Skilled practitioners will also appreciate that carbon monoxide compositions need not be administered to a patient by the same individual who diagnosed the patient (or prescribed the carbon monoxide composition for the patient). Carbon monoxide compositions can be administered (and/or administration can be supervised), e.g., by the diagnosing and/or prescribing individual, and/or any other individual, including the patient her/himself (e.g., where the patient is capable of self-administration).

The methods of the present invention can also be used to inhibit unwanted (e.g., detrimental) angiogenesis in a patient and to treat angiogenesis dependent/associated conditions associated therewith. As used herein, the term “angiogenesis” means the generation of new blood vessels in a tissue or organ. An “angiogenesis dependent/associated condition” includes any process or condition that is dependent upon or associated with angiogenesis. The term includes conditions that involve cancer, as well as those that do not. Angiogenesis dependent/associated conditions can be associated with (e.g., arise from) unwanted angiogenesis, as well as with wanted (e.g., beneficial) angiogenesis. The term includes, e.g., solid tumors; tumor metastasis; benign tumors, e.g., hemangiomas, acoustic neuromas, neurofibromas, trachomas, and pyogenic granulomas; rheumatoid arthritis, lupus, and other connective tissue disorders; psoriasis; rosacea; ocular angiogenic diseases, e.g., diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. Other processes in which angiogenesis is involved include reproduction and wound healing. Because of its anti-VEGF properties, CO can also be useful in the treatment of diseases of excessive or abnormal stimulation of endothelial cells. Such diseases include, e.g., intestinal adhesions, atherosclerosis, scleroderma, and hypertrophic scars, e.g., keloids, as well as endothelial cell cancers that are sensitive to VEGF stimulation.

Amounts of CO effective to treat cancer, angiogenesis dependent/associated conditions (e.g., conditions other than cancer), or to inhibit unwanted angiogenesis in a patient, can be administered to (or prescribed for) a patient, e.g., by a physician or veterinarian, on the day the patient is diagnosed as suffering any of these disorders or conditions, or as having any risk factor associated with an increased likelihood that the patient will develop such disorder(s) or condition(s) (e.g., the patient has recently been, is being, or will be exposed to a carcinogen(s)). Patients can inhale CO at concentrations ranging from 10 ppm to 1000 ppm, e.g., about 100 ppm to about 800 ppm, about 150 ppm to about 600 ppm, or about 200 ppm to about 500 ppm. Preferred concentrations include, e.g., about 30 ppm, 50 ppm, 75 ppm, 100 ppm, 125 ppm, 200 ppm, 250 ppm, 500 ppm, 750 ppm, or about 1000 ppm. CO can be administered to the patient intermittently or continuously. CO can be administered for at least about 1, 2, 4, 6, 8, 10, 12, 14, 18, or 20 days, or greater than 20 days, e.g., 1 2, 3, 5, or 6 months, or until the patient no longer exhibits symptoms of the condition or disorder, or until the patient is diagnosed as no longer being at risk for the condition or disorder. In a given day, CO can be administered continuously for the entire day, or intermittently, e.g., a single whiff of CO per day (where a high concentration is used), or for up to 23 hours per day, e.g., up to 20, 15, 12, 10, 6, 3, or 2 hours per day, or up to 1 hour per day.

If the patient needs to be treated with chemotherapy, radiation therapy, immunotherapy, gene therapy, and/or surgery (e.g., because prescribed by a physician or veterinarian), the patient can be treated with CO (e.g., a gaseous CO composition) before, during, and/or after administration of the chemotherapy, radiation therapy, and/or surgery. For example, with regard to chemotherapy, immunotherapy, gene therapy, and radiation therapy, CO can be administered to the patient, intermittently or continuously, starting 0 to 20 days before the chemotherapy, immunotherapy, gene therapy, or radiation therapy is administered (and where multiple doses are given, before each individual dose), e.g., starting at least about 30 minutes, e.g., about 1, 2, 3, 5, 7, or 10 hours, or about 1, 2, 4, 6, 8, 10, 12, 14, 18, or 20 days, or greater than 20 days, before the administration. Alternatively or in addition, CO can be administered to the patient concurrent with administration of chemotherapy, immunotherapy, gene therapy, or radiation therapy. Alternatively or in addition, CO can be administered to the patient after administration of chemotherapy, immunotherapy, gene therapy, or radiation therapy, e.g., starting immediately after administration, and continuing intermittently or continuously for about 1, 2, 3, 5, 7, or 10 hours, or about 1, 2, 5, 8, 10, 20, 30, 50, or 60 days, one year, indefinitely, or until a physician determines that administration of the CO is no longer necessary.

With regard to surgical procedures, CO can be administered systemically or locally to a patient prior to, during, and/or after a surgical procedure is performed. Patients can inhale CO at concentrations ranging from 10 ppm to 1000 ppm, e.g., about 100 ppm to about 800 ppm, about 150 ppm to about 600 ppm, or about 200 ppm to about 500 ppm. Preferred concentrations include, e.g., about 30 ppm, 50 ppm, 75 ppm, 100 ppm, 125 ppm, 200 ppm, 250 ppm, 500 ppm, 750 ppm, or about 1000 ppm. CO can be administered to the patient intermittently or continuously, for 1 hour, 2, hours, 3 hours, 4 hours, 6, hours, 12 hours, or about 1, 2, 4, 6, 8, 10, 12, 14, 18, or 20 days, or greater than 20 days, before the procedure. It can be administered in the time period immediately prior to the surgery and optionally continue through the procedure, or the administration can cease at least 15 minutes before the surgery begins (e.g., at least 30 minutes, 1 hour, 2 hours 3 hours, 6 hours, or 24 hours before the surgery begins. Alternatively or in addition, CO can be administered to the patient during the procedure, e.g., by inhalation and/or topical administration. Alternatively or in addition, CO can be administered to the patient after the procedure, e.g., starting immediately after completion of the procedure, and continuing for about 1, 2, 3, 5, 7, or 10 hours, or about 1, 2, 5, 8, 10, 20, 30, 50, or 60 days, 1 year, indefinitely, or until the patient no longer suffers from, or is at risk for, cancer after the completion of the procedure.

Preparation of Gaseous Compositions

A carbon monoxide composition may be a gaseous carbon monoxide composition. Compressed or pressurized gas useful in the methods of the invention can be obtained from any commercial source, and in any type of vessel appropriate for storing compressed gas. For example, compressed or pressurized gases can be obtained from any source that supplies compressed gases, such as oxygen, for medical use. The term “medical grade” gas, as used herein, refers to gas suitable for administration to patients as defined herein. The pressurized gas including CO used in the methods of the present invention can be provided such that all gases of the desired final composition (e.g., CO, He, NO, CO₂, O₂, N₂) are in the same vessel, except that NO and O₂ cannot be stored together. Optionally, the methods of the present invention can be performed using multiple vessels containing individual gases. For example, a single vessel can be provided that contains carbon monoxide, with or without other gases, the contents of which can be optionally mixed with room air or with the contents of other vessels, e.g., vessels containing oxygen, nitrogen, carbon dioxide, compressed air, or any other suitable gas or mixtures thereof.

Gaseous compositions administered to a patient according to the present invention typically contain 0% to about 79% by weight nitrogen, about 21% to about 100% by weight oxygen and about 0.0000001% to about 0.3% by weight (corresponding to about 1 ppb or 0.001 ppm to about 3,000 ppm) carbon monoxide. Preferably, the amount of nitrogen in the gaseous composition is about 79% by weight, the amount of oxygen is about 21% by weight and the amount of carbon monoxide is about 0.0001% to about 0.25% by weight, preferably at least about 0.001%, e.g., at least about 0.005%, 0.01%, 0.02%, 0.025%, 0.03%, 0.04%, 0.05%, 0.06%, 0.08%, 0.10%, 0.15%, 0.20%, 0.22%, or 0.24% by weight. Preferred ranges of carbon monoxide include 0.005% to about 0.24%, about 0.01% to about 0.22%, about 0.015% to about 0.20%, about 0.08% to about 0.20%, and about 0.025% to about 0.1% by weight. It is noted that gaseous carbon monoxide compositions having concentrations of carbon monoxide greater than 0.3% (such as 1% or greater) may be used for short periods (e.g., one or a few breaths), depending upon the application.

A gaseous carbon monoxide composition may be used to create an atmosphere that comprises carbon monoxide gas. An atmosphere that includes appropriate levels of carbon monoxide gas can be created, for example, by providing a vessel containing a pressurized gas comprising carbon monoxide gas, and releasing the pressurized gas from the vessel into a chamber or space to form an atmosphere that includes the carbon monoxide gas inside the chamber or space. Alternatively, the gases can be released into an apparatus that culminates in a breathing mask or breathing tube, thereby creating an atmosphere comprising carbon monoxide gas in the breathing mask or breathing tube, ensuring the patient is the only person in the room exposed to significant levels of carbon monoxide.

Carbon monoxide levels in an atmosphere or a ventilation circuit can be measured or monitored using any method known in the art. Such methods include electrochemical detection, gas chromatography, radioisotope counting, infrared absorption, colorimetry, and electrochemical methods based on selective membranes (see, e.g., Sunderman et al., Clin. Chem. 28:2026-2032, 1982; Ingi et al., Neuron 16:835-842, 1996). Sub-parts per million carbon monoxide levels can be detected by, e.g., gas chromatography and radioisotope counting. Further, it is known in the art that carbon monoxide levels in the sub-ppm range can be measured in biological tissue by a midinfrared gas sensor (see, e.g., Morimoto et al., Am. J. Physiol. Heart. Circ. Physiol 280:H482-H488, 2001). Carbon monoxide sensors and gas detection devices are widely available from many commercial sources.

Preparation of Liquid Compositions

A carbon monoxide composition may also be a liquid carbon monoxide composition. A liquid can be made into a carbon monoxide composition by any method known in the art for causing gases to become dissolved in liquids. For example, the liquid can be placed in a so-called “CO₂ incubator” and exposed to a continuous flow of carbon monoxide, preferably balanced with carbon dioxide, until a desired concentration of carbon monoxide is reached in the liquid. As another example, carbon monoxide gas can be “bubbled” directly into the liquid until the desired concentration of carbon monoxide in the liquid is reached. The amount of carbon monoxide that can be dissolved in a given aqueous solution increases with decreasing temperature. As still another example, an appropriate liquid may be passed through tubing that allows gas diffusion, where the tubing runs through an atmosphere comprising carbon monoxide (e.g., utilizing a device such as an extracorporeal membrane oxygenator). The carbon monoxide diffuses into the liquid to create a liquid carbon monoxide composition.

It is likely that such a liquid composition intended to be introduced into a living animal will be at or about 37° C. at the time it is introduced into the animal.

The liquid can be any liquid known to those of skill in the art to be suitable for administration to patients (see, for example, Oxford Textbook of Surgery, Morris and Malt, Eds., Oxford University Press (1994)). In general, the liquid will be an aqueous solution. Examples of solutions include Phosphate Buffered Saline (PBS), Celsior™, Perfadex™, Collins solution, citrate solution, and University of Wisconsin (UW) solution (Oxford Textbook of Surgery, Morris and Malt, Eds., Oxford University Press (1994)). In one embodiment of the present invention, the liquid is Ringer's Solution, e.g., lactated Ringer's Solution, or any other liquid that can be used infused into a patient. In another embodiment, the liquid includes blood, e.g., whole blood. The blood can be completely or partially saturated with carbon monoxide.

Any suitable liquid can be saturated to a set concentration of carbon monoxide via gas diffusers. Alternatively, pre-made solutions that have been quality controlled to contain set levels of carbon monoxide can be used. Accurate control of dose can be achieved via measurements with a gas permeable, liquid impermeable membrane connected to a carbon monoxide analyzer. Solutions can be saturated to desired effective concentrations and maintained at these levels.

Treatment of Patients with Carbon Monoxide Compositions

A patient can be treated with a carbon monoxide composition by any method known in the art of administering gases and/or liquids to patients. Carbon monoxide compositions can be prescribed for and/or administered to a patient diagnosed with, or determined to be at risk for, e.g., cancer. The present invention contemplates the systemic administration of liquid or gaseous carbon monoxide compositions to patients (e.g., by inhalation and/or ingestion), and the topical administration of the compositions to the patient's organs in situ (e.g., by ingestion, insufflation, and/or introduction into the abdominal cavity). The compositions can be administered and/or supervised by any person, e.g., a health-care professional, veterinarian, or caretaker (e.g., an animal (e.g., dog or cat) owner), depending upon the patient to be treated, and/or by the patient him/herself, if the patient is capable of doing so.

Systemic Delivery of Carbon Monoxide

Gaseous carbon monoxide compositions can be delivered systemically to a patient, e.g., a patient diagnosed with or determined to be at risk for cancer. Gaseous carbon monoxide compositions are typically administered by inhalation through the mouth or nasal passages to the lungs, where the carbon monoxide is readily absorbed into the patient's bloodstream. The concentration of active compound (CO) utilized in the therapeutic gaseous composition will depend on absorption, distribution, inactivation, and excretion (generally, through respiration) rates of the carbon monoxide as well as other factors known to those of skill in the art. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the invention. Treatments can be monitored and CO dosages can be adjusted to ensure optimal treatment of the patient. Acute, sub-acute and chronic administration of carbon monoxide are contemplated by the present invention, depending upon, e.g., the severity or persistence of the disorder in the patient. Carbon monoxide can be delivered to the patient for a time (including indefinitely) sufficient to treat the condition and exert the intended pharmacological or biological effect.

The following are examples of some methods and devices that can be utilized to administer gaseous carbon monoxide compositions to patients.

Ventilators

Medical grade carbon monoxide (concentrations can vary) can be purchased mixed with air or another oxygen-containing gas in a standard tank of compressed gas (e.g., 21% O₂, 79% N₂). It is non-reactive, and the concentrations that are required for the methods of the present invention are well below the combustible range (10% in air). In a hospital setting, the gas presumably will be delivered to the bedside where it will be mixed with oxygen or house air in a blender to a desired concentration in ppm (parts per million). The patient will inhale the gas mixture through a ventilator, which will be set to a flow rate based on patient comfort and needs. This is determined by pulmonary graphics (i.e., respiratory rate, tidal volumes, etc.). Fail-safe mechanism(s) to prevent the patient from unnecessarily receiving greater than desired amounts of carbon monoxide can be designed into the delivery system. The patient's carbon monoxide level can be monitored by studying (1) carboxyhemoglobin (COHb), which can be measured in venous blood, and (2) exhaled carbon monoxide collected from a side port of the ventilator. Carbon monoxide exposure can be adjusted based upon the patient's health status and on the basis of the markers. If necessary, carbon monoxide can be washed out of the patient by switching to 100% O₂ inhalation. Carbon monoxide is not metabolized; thus, whatever is inhaled will ultimately be exhaled except for a very small percentage that is converted to CO₂. Carbon monoxide can also be mixed with any level of O₂ to provide therapeutic delivery of carbon monoxide without consequential hypoxic conditions.

Face Mask and Tent

A carbon monoxide-containing gas mixture is prepared as above to allow inhalation by the patient using a facemask or tent. The concentration inhaled can be changed and can be washed out by simply switching over to 100% O₂. Monitoring of carbon monoxide levels would occur at or near the mask or tent with a fail-safe mechanism that would prevent too high of a concentration of carbon monoxide from being inhaled.

Portable Inhaler

Compressed carbon monoxide can be packaged into a portable inhaler device and inhaled in a metered dose, for example, to permit intermittent treatment of a recipient who is not in a hospital setting. Different concentrations of carbon monoxide could be packaged in the containers. The device could be as simple as a small tank (e.g., under 5 kg) of appropriately diluted CO with an on-off valve and a tube from which the patient takes a whiff of CO according to a standard regimen or as needed.

Intravenous Artificial Lung

An artificial lung (a catheter device for gas exchange in the blood) designed for O₂ delivery and CO₂ removal can be used for carbon monoxide delivery. The catheter, when implanted, resides in one of the large veins and would be able to deliver carbon monoxide at given concentrations either for systemic delivery or at a local site. The delivery can be a local delivery of a high concentration of carbon monoxide for a short period of time at the site of the tumor (this high concentration would rapidly be diluted out in the bloodstream), or a relatively longer exposure to a lower concentration of carbon monoxide (see, e.g., Hattler et al., Artif. Organs 18(11):806-812, 1994; and Golob et al., ASAIO J. 47(5):432-437, 2001).

Normobaric Chamber

In certain instances, it would be desirable to expose the whole patient to carbon monoxide. The patient would be inside an airtight chamber that would be flooded with carbon monoxide (at a level that does not endanger the patient, or at a level that poses an acceptable risk without the risk of bystanders being exposed). Upon completion of the exposure, the chamber could be flushed with air (e.g., 21% O₂, 79% N₂), and samples could be analyzed by carbon monoxide analyzers to ensure no carbon monoxide remains before allowing the patient to exit the exposure system.

Systemic Delivery of Liquid CO Compositions

The present invention further contemplates that aqueous solutions comprising carbon monoxide can be created for systemic delivery to a patient, e.g., for oral delivery and/or by infusion into the patient, e.g., intravenously, intra-arterially, intraperitoneally, and/or subcutaneously. For example, liquid CO compositions, such as CO-saturated Ringer's Solution, can be infused into a patient suffering from or at risk for cancer. Alternatively or in addition, CO-partially or completely saturated whole (or partial) blood can be infused into the patient.

The present invention also contemplates that agents capable of delivering doses of gaseous CO compositions or liquid CO compositions can be utilized (e.g., CO-releasing gums, creams, ointments, lozenges, or patches).

Topical Treatment of Organs with Carbon Monoxide

Alternatively or in addition, carbon monoxide compositions can be applied directly to organs, e.g., the skin and internal organs. Gaseous compositions can be applied directly to the interior and/or exterior of the patient's body to treat the patient's organs. A gaseous composition can be directly applied to the internal organs of a patient by any method known in the art for insufflating gases into a patient. For example, gases, e.g., carbon dioxide, are often insufflated into the abdominal cavity of patients to facilitate examination during laproscopic procedures (see, e.g., Oxford Textbook of Surgery, Morris and Malt, Eds., Oxford University Press (1994)). Skilled practitioners will appreciate that similar procedures could be used to administer carbon monoxide compositions directly to an internal organ of a patient. The skin can be treated topically with a gaseous composition by, for example, exposing the affected skin to the gaseous composition in a normobarometric chamber (described above), and/or by blowing the carbon monoxide composition directly onto the skin. If the patient does not inhale the gas, the concentration of CO in the gaseous composition could be as high as desired, e.g., over 0.25% and up to about 100%.

Liquid carbon monoxide compositions can also be administered topically to a patient's organs. Liquid forms of the compositions can be administered by any method known in the art for administering liquids to patients. As with gaseous compositions, liquid compositions can be applied directly to the interior and/or exterior of the body to treat a patient's organs. For example, the liquid compositions can be administered orally, e.g., by causing the patient to ingest an encapsulated or unencapsulated dose of the aqueous carbon monoxide composition. As another example, liquids, e.g., saline solutions containing dissolved CO, can be injected into the abdominal cavity of patients during laproscopic procedures. Alternatively or in addition, in situ exposures or organs can be performed by any method known in the art, e.g., by in situ flushing of the organ with a liquid carbon monoxide composition during surgery (see Oxford Textbook of Surgery, Morris and Malt, Eds., Oxford University Press (1994)). The skin can be treated topically with a liquid composition by, for example, injecting the liquid composition into the skin. As a further example, the skin can be treated topically by applying the liquid composition directly to the surface of the skin, e.g., by pouring or spraying the liquid onto the skin and/or by submerging the skin in the liquid composition. Other externally-accessible surfaces such as the eye, mouth, throat, vagina, cervix, urinary tract, colon, and anus can be similarly treated topically with the liquid compositions.

Use of Hemoxygenase-1 and Other Compounds

Also contemplated by the present invention is the induction, expression, and/or administration of hemeoxygenase-1 (HO-1) in conjunction with administration of carbon monoxide. HO-1 can be provided to a patient by inducing or expressing HO-1 in the patient, or by administering exogenous HO-1 directly to the patient. As used herein, the term “induce(d)” means to cause increased production of a protein, e.g., HO-1, in isolated cells or the cells of a tissue, organ or animal using the cells' own endogenous (e.g., non-recombinant) gene that encodes the protein.

HO-1 can be induced in a patient by any method known in the art. For example, production of HO-1 can be induced by hemin, by iron protoporphyrin, or by cobalt protoporphyrin. A variety of non-heme agents including heavy metals, cytokines, hormones, nitric oxide (NO), COCl₂, endotoxin and heat shock are also strong inducers of HO-1 expression (Otterbein et al., Am. J. Physiol. Lung Cell Mol. Physiol. 279:L1029-L1037, 2000; Choi et al., Am. J. Respir. Cell Mol. Biol. 15:9-19, 1996; Maines, Annu. Rev. Pharmacol. Toxicol. 37:517-554, 1997; and Tenhunen et al., J. Lab. Clin. Med. 75:410-421, 1970). HO-1 is also highly induced by a variety of agents and conditions that create oxidative stress, including hydrogen peroxide, glutathione depletors, UV irradiation and hyperoxia (Choi et al., Am. J. Respir. Cell Mol. Biol. 15: 9-19, 1996; Maines, Annu. Rev. Pharmacol. Toxicol. 37:517-554, 1997; and Keyse et al., Proc. Natl. Acad. Sci. USA 86:99-103, 1989). A “pharmaceutical composition comprising an inducer of HO-1” means a pharmaceutical composition containing any agent capable of inducing HO-1 in a patient, e.g., any of the agents described above, e.g., NO, hemin, iron protoporphyrin, and/or cobalt protoporphyrin.

HO-1 expression in a cell can be increased via gene transfer. As used herein, the term “express(ed)” means to cause increased production of a protein, e.g., HO-1 or ferritin, in isolated cells or the cells of a tissue, organ or animal using an exogenously administered gene (e.g., a recombinant gene). The HO-1 or ferritin is preferably of the same species (e.g., human, mouse, rat, etc.) as the patient, in order to minimize any immune reaction. Expression could be driven by a constitutive promoter (e.g., cytomegalovirus promoters) or a tissue-specific promoter (e.g., milk whey promoter for mammary cells or albumin promoter for liver cells). An appropriate gene therapy vector (e.g., retroviruses, adenoviruses, adeno associated viruses (AAV), pox (e.g., vaccinia) viruses, human immunodeficiency virus (HIV), the minute virus of mice, hepatitis B virus, influenza virus, Herpes Simplex Virus-1, and lentiviruses) encoding HO-1 or ferritin would be administered to the patient orally, by inhalation, or by injection at a location appropriate for treatment of a disorder or condition described herein. Particularly preferred is local administration directly to the site of the condition, e.g., to a tumor and/or an organ in which the tumor has or is beginning to develop. Similarly, plasmid vectors encoding HO-1 or apoferritin can be administered, e.g., as naked DNA, in liposomes, or in microparticles.

Further, exogenous HO-1 protein can be directly administered to a patient by any method known in the art. Exogenous HO-1 can be directly administered in addition to, or as an alternative, to the induction or expression of HO-1 in the patient as described above. The HO-1 protein can be delivered to a patient, for example, in liposomes, and/or as a fusion protein, e.g., as a TAT-fusion protein (see, e.g., Becker-Hapak et al., Methods 24, 247-256 (2001)).

Alternatively or in addition, any of the products of metabolism by HO-1, e.g., bilirubin, biliverdin, iron, and/or ferritin, can be administered to a patient in conjunction with carbon monoxide in order to prevent or treat the condition or disorder. Further, the present invention contemplates that iron-binding molecules other than ferritin, e.g., desferoxamine (DFO), iron dextran, and/or apoferritin, can be administered to the patient. Further still, the present invention contemplates that enzymes (e.g., biliverdin reductase) that catalyze the breakdown any of these products can be inhibited to create/enhance the desired effect. Any of the above can be administered, e.g., orally, intravenously, intraperitoneally, or topically.

The present invention contemplates that compounds that release CO into the body after administration of the compound (e.g., CO-releasing compounds, e.g., photoactivatable CO-releasing compounds), e.g., dimanganese decacarbonyl, tricarbonyldichlororuthenium(II) dimer, and methylene chloride (e.g., at a dose of between 400 to 600 mg/kg, e.g., about 500 mg/kg), can also be used in the methods of the present invention, as can carboxyhemoglobin and CO-donating hemoglobin substitutes.

The above can be administered to a patient in any way, e.g., by oral, intraperitoneal, intravenous, or intraarterial administration. Any of the above compounds can be administered to the patient locally and/or systemically, and in any combination.

Combination Therapy

Also contemplated by the present invention is administration of CO to a patient in conjunction with at least one other treatment, e.g., chemotherapy, radiation therapy, immunotherapy, gene therapy, and/or surgery, to treat conditions and disorders described herein (e.g., cancer). For example, CO can be administered to a patient using any method described herein in combination with surgery to remove cancerous tissue from the patient. Alternatively or in addition, treatments described herein can be administered in combination with chemotherapy. Chemotherapy can involve administration of any of the following classes of compounds: alkylating agents, antimetabolites, e.g., folate antagonists, purine antagonists and/or pyrimidine antagonists; spindle poisons, e.g., vincas (e.g., paclitaxel) and podophillotoxins; antibiotics, e.g., doxorubicin, bleomycin and/or mitomycin; nitrosoureas; inorganic ions, e.g., cisplatin; biologic response modifiers, e.g., tumor necrosis factor-α (TNF-α) and interferon; enzymes, e.g., asparaginase; protein toxins conjugated to targeting moieties; antisense molecules; and hormones, e.g, tomoxifen, leuprolide, flutamide, and megestrol. Alternatively or in addition, treatments described herein can be administered in combination with radiation therapy, e.g., using γ-radiation, neutron beams, electron beams, and/or radioactive isotopes. Alternatively or in addition, treatments described herein can be administered to patients in combination with immunotherapy, e.g., the administration of specific effector cells, tumor antigens, and/or antitumor antibodies. Alternatively or in addition, treatments described herein can be administered to patients in combination with gene therapy, e.g., the administration of DNA encoding tumor antigens and/or cytokines. Methods for treating cancer, e.g., surgery, chemotherapy, immunotherapy, and radiotherapy, are more fully described in the The Merck Manual of Diagnosis and Therapy, 17^{th} Edition, Section 11, Chapters 143 and 144, the contents of which are expressly incorporated herein by reference in their entirety.

The invention is illustrated in part by the following examples, which are not to be taken as limiting the invention in any way.

### EXAMPLE 1

### CO Inhibits the Growth of Tumors and Cancer Cells Both In Vivo and In Vitro, and Inhibits Angiogenesis

Animals

For human tumor studies, female SCID mice (weighing 20 to 30 g) were purchased from Taconic (White Plains, N.Y.) and allowed to acclimate for 1 week prior to experimentation. For murine tumor and matrigel studies, male CBA and C57B1/6 mice (weighing 25 to 30 g) were purchased form Jackson Labs (Bar Harbor, Me.) and also were allowed to acclimate for 1 week prior to experimentation.

Cell Lines

A human adenocarcinoma cell line designated A549, a murine mesothelioma cell line designated AC29, and a human colon cancer cell line designated HCT were utilized for the studies described herein.

CO Exposure

Cell cultures and mice were exposed to CO at a concentration of 250 ppm. Briefly, 1% CO in air was mixed with air (21% oxygen) in a stainless steel mixing cylinder and then directed into a 3.70 ft³ glass exposure chamber at a flow rate of 12 L/min. A CO analyzer (Interscan, Chatsworth, Calif.) was used to measure CO levels continuously in the chamber. CO concentrations were maintained at 250 ppm at all times. Cell cultures and mice were placed in the exposure chamber as required.

General Procedures

ELISA kits for VEGF levels were purchased from R&D Systems and used according to the manufacturer's directions.

Immunoblotting was performed to investigate protein expression by standard methods known in the art. Antibodies were purchased from Santa Cruz, StressGen and Cell Signaling.

For [³H] thymidine incorporation studies, cells were serum-starved overnight and then stimulated with 20% serum containing 5 mCi/ml [³H] thymidine (New England Nuclear, Boston, Mass.). [³H] thymidine incorporation was measured by scintillation spectroscopy. CO inhibits the growth of cancer cells in vitro Human and mouse cancer cell lines were used to investigate the effect of CO on growth rates of the cells in culture. Human adenocarcinoma cells (A549), mouse mesothelioma cells (AC29), and A549 and AC29 cells transformed with the heme oxygenase-1 (HO-1) gene (which causes the cells to overexpress HO-1) were exposed to low levels of CO (250 ppm) in culture. Four-day growth curves were generated. Cells exposed to CO plus air showed growth patterns similar to cells that overexpress HO-1, e.g., a >40% reduction in cell number by three days, compared to controls (data not shown). These reduced numbers were not due to toxicity because confluency was eventually achieved, albeit at a significantly reduced rate.

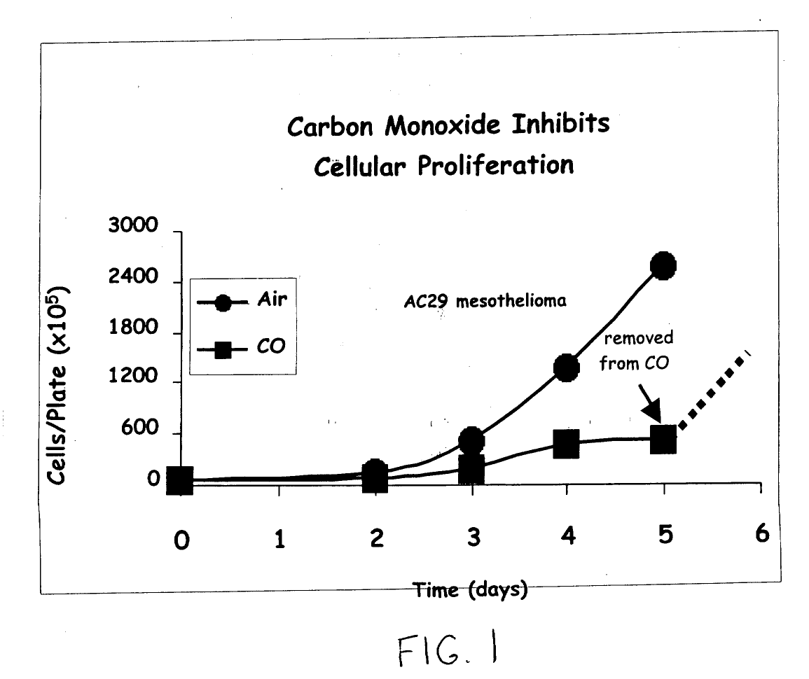

FIG. 1 is a six-day growth curve which illustrates that CO inhibits the proliferation of AC29 murine mesothelioma cancer cells. At day 5, CO-exposed cell cultures where removed from the CO-containing atmosphere, and thereafter were observed to proliferate at a normal rate.

CO and HO-1 Inhibit Tumor Growth In Vivo

Mouse models of tumor growth were used to evaluate the ability of HO-1 and CO to inhibit tumor growth. Three models of tumor growth in mice were utilized.

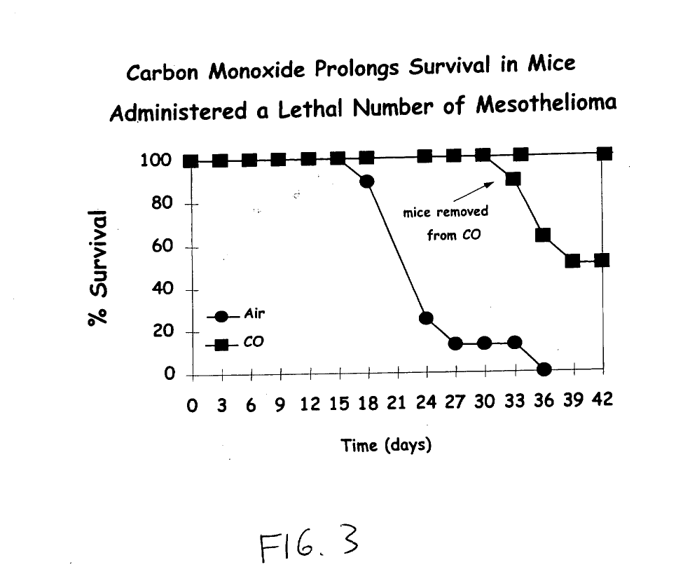

The first was a mesothelioma (AC29) model, wherein CBA mice were injected with 1×10⁶ AC29 cells intraperitoneally and monitored for survival when continuously exposed to air or to an atmosphere containing 250 ppm CO for a period of six weeks. As can be seen in FIG. 3, mice exposed to CO lived longer than mice exposed to air alone. The survival rate of the CO-exposed mice was increased by greater than 90% as compared to air-exposed mice. The arrow shown in FIG. 3 denotes a time point at which half of the CO-exposed mice were removed from the CO chamber. Half of the mice were removed at that time to determine whether the effects of CO on mouse survival require continuous exposure to CO. A significant number of mice (50%, p<0.02) that were removed from the CO-containing atmosphere remained alive at the end of the experiment, whereas all air treated mice died by day 36. The number of mice in each group was 12 to 20 animals.

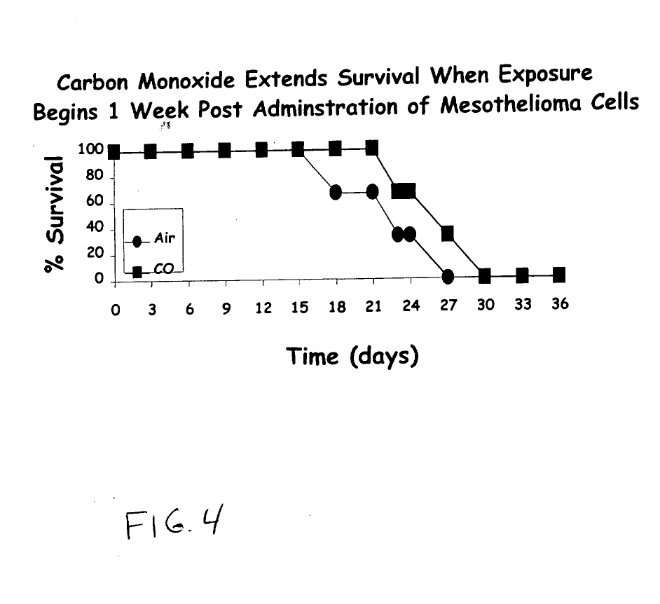

In another experiment, it was shown that CO-exposed mice survived for greater than 65 days (data not shown). Further, as illustrated by FIG. 4, CO exposure prolonged the lives of mice even when CO treatment was delayed until one week after injection of mesothelioma cells.

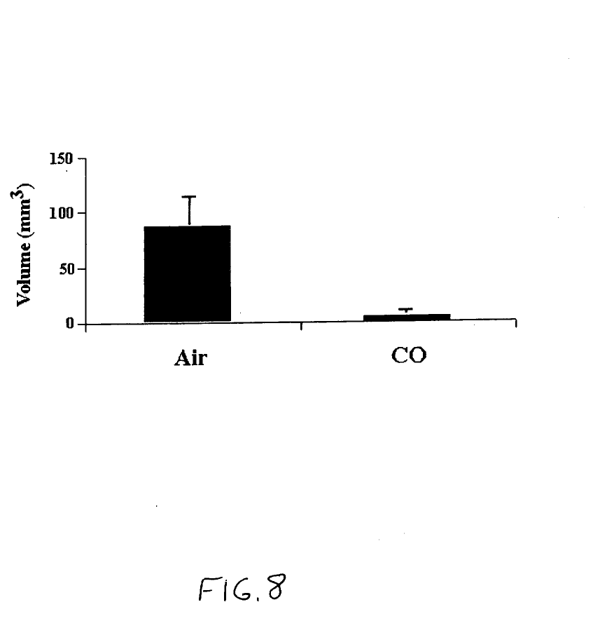

The second model was an adenocarcinoma (A549) model, wherein SCID mice were injected with 1×10⁶ A549 cells subcutaneously. These animals were continuously exposed to air or to 250 ppm of CO for a period of six weeks. After the six-week period, the volume of the tumors that developed in the mice was evaluated. As can be seen in FIG. 8, tumor volume is significantly less (greater than 50% less) in CO-exposed mice as compared to air-exposed mice.

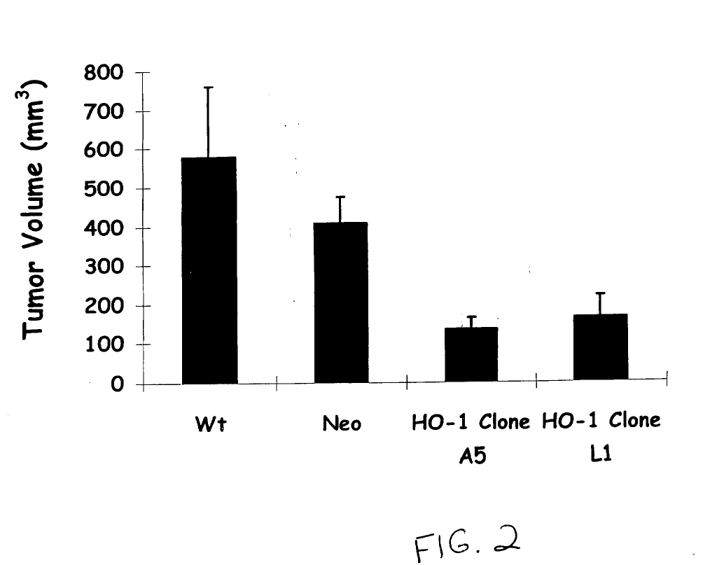

The third model was also an A549 model, wherein mice were injected with A549 cells transformed to overexpress HO-1 (HO-1 clones A5 and L1). After the six-week period, the size and volume of tumors that developed in the mice were evaluated. As illustrated in FIG. 2, those mice injected with the A549 HO-1 cells showed reduced tumor volume versus vector (Neo) and wild type (Wt) cell controls. The inhibitory effect of overexpression of HO-1 on tumor growth was shown to be reversible upon administering to the mice a dose of tin protoporphyrin (50 μmol/kg, subcutaneously (s.c.)), which is an inhibitor of HO-1 (data not shown). Using Western blot analysis, the relative decrease in volume was determined to correlate with a relative decrease in expression of cyclin D1, a protein involved in the regulation of cell growth (data not shown). Cyclin D1 is highly expressed in growing cells, and a decrease in cyclin D1 expression indicates that cell growth is inhibited.

Mechanisms of CO Inhibition of Cancer Cell Proliferation

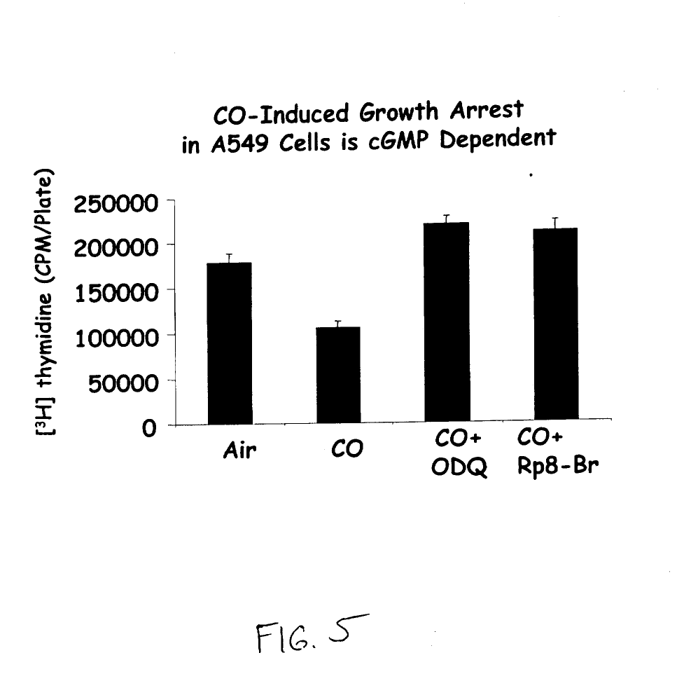

The cellular mechanisms by which CO causes inhibition were also investigated. To investigate whether CO-induced growth arrest is CGMP dependant, A549 cells were exposed to air, CO, CO+ODQ, or CO+Rp-8-BR. ODQ is a compound that selectively inhibits guanylate cyclase, and Rp-8-Br is an inactive analog of cGMP that competitively inhibits the cGMP signaling pathway. The ability of the cells to proliferate was determined by measuring the uptake of [3H] thymidine by the cells (FIG. 5). Cells were exposed to CO (250 ppm) for 3 hours prior to the addition of serum and [3H] thymidine (5 mCi/ml). After the addition of serum and [3H] thymidine, cells were maintained in CO for 24 hours. Cells were then rinsed, fixed and examined by scintillation spectroscopy. As can be seen in FIG. 5, A549 cells exposed to air, CO+ODQ, or CO+Rp8-BR exhibited greater uptake of [³H] thymidine as compared to cells exposed to CO alone. These data indicate that CO-induced growth arrest is cGMP dependant.

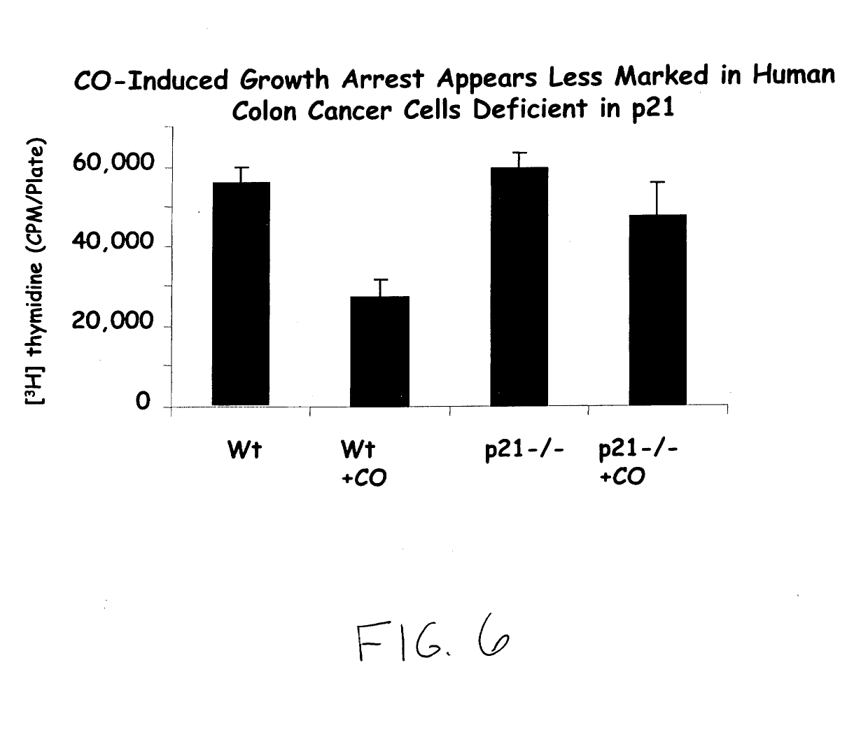

Wild-type (Wt) HTC cells and HTC cells deficient in p21 (p21−/−), a gene known to control cell growth, were exposed to air or CO to determine whether p21 is involved in CO-induced growth arrest (FIG. 6). As indicated by [³H] thymidine uptake, CO-induced growth arrest appears less marked in HTC cells that are deficient in p21.

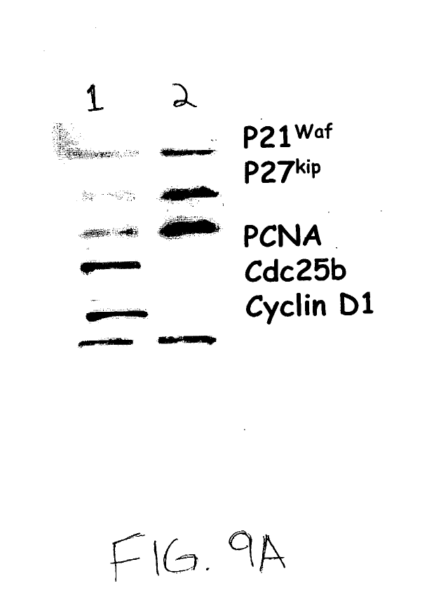

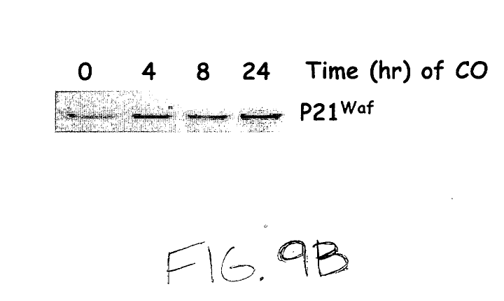

To investigate CO-induced changes in the expression of various growth/cell cycle proteins in cancer cells, A549 cells were exposed for 24 hours to air or CO (250 ppm). After this period of exposure, cell lysates were collected from the cells and changes in protein expression in the lysates were examined by immunoblot. It was observed that CO caused changes in expression of p21, p27, proliferating cell nuclear antigen (PCNA), Cdc25b, and cyclin D1, all of which are involved in cell growth and proliferation (FIGS. 9A and 9B).

CO appears to inhibit cell proliferation at the G1/S phase of the cell cycle which is cGMP-dependent. The mechanism of CO action appears to involve modulation of p21, p27, cyclin D1, PCNA, Cdc25b and p38 MAP kinase signal transduction (upregulated) (data not shown).

CO Inhibits Vascular Endothelial Growth Factor (VEGF) Production and Angiogenesis

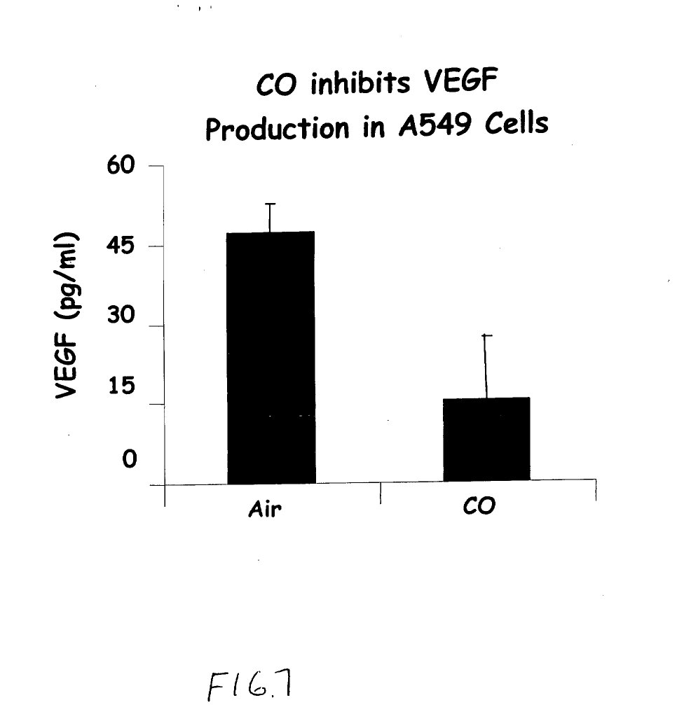

Whether CO inhibits production of VEGF, a growth factor that contributes to angiogenesis by promoting blood vessel growth, was investigated. A549 cells were exposed to air or CO plus air for 24 to 48 hours in vitro, and VEGF production by A549 cells was detected using an enzyme-linked immunosorbent assay (ELISA). As illustrated in FIG. 7, cells exposed to CO plus air produced substantially less VEGF than cells exposed to air alone.

The effect of CO (250 ppm) on angiogenesis was investigated using a Matri gel in vitro angiogenesis assay. A solubilized basement membrane matrix (Matrigel™) containing 20 ng/ml growth factor (FGF) and heparin was implanted under the skin of C57/B16 mice. The mice were then exposed to air or CO in air for two weeks. After the two-week period, the Matrigel™ was removed and examined. Mice that were exposed to air alone exhibited the beginning stages of angiogenesis, while mice exposed to CO in air exhibited no new blood vessel growth (data not shown).

In a separate experiment, Matrigel™ deposits containing 20 ng/ml growth factor (FGF) and heparin were implanted under the skin of C57/B16 mice, and the mice were exposed to air or CO (250 ppm) in air for 21 days. Photomicrographs of hematoxylin- and eosin-stained paraffin sections from the resected subcutaneous FGF-Matrigel deposits were prepared. Prominent angiogenesis was evident in deposits from air-exposed mice, as was a front of infiltrating vascular cells organizing into blood filled micro-capillaries (data not shown). No angiogenesis was evident in deposits from CO-treated mice, and there was a paucity of cellularity and blood in these deposits.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating naturally arising cancer in a patient, comprising:
administering to a patient diagnosed as suffering from or at risk for naturally arising cancer a therapeutically effective amount of a composition comprising carbon monoxide.

2. The method of claim 1, wherein the composition is in gaseous form and is administered to the patient via inhalation.

3. The method of claim 1, wherein the composition is in gaseous form and is administered topically to an organ of the patient other than the patient's lungs.

4. The method of claim 1, wherein the composition in is gaseous form and is administered to the abdominal cavity of the patient.

5. The method of claim 1, wherein the composition is in liquid form and is administered to the patient orally.

6. The method of claim 1, wherein the composition is in liquid form and is administered topically to an organ of the patient.

7. The method of claim 1, wherein the composition is in liquid form and is administered to the abdominal cavity of the patient.

8. The method of claim 1, wherein the patient has previously undergone surgery to remove cancerous tissue.

9. The method of claim 1, further comprising performing surgery on the patient to remove cancerous tissue.

10. The method of claim 1, wherein the administration takes place during surgery to remove cancerous tissue.

11. The method of claim 1, wherein the patient has previously undergone chemotherapy or radiation therapy.

12. The method of claim 1, further comprising administering chemotherapy or radiation therapy to the patient to treat the cancer.

13. The method of claim 1, wherein the administration takes place during chemotherapy or radiation therapy to treat the cancer.

14. The method of claim 1, wherein the patient is a human.

15. The method of claim 1, wherein the cancer is cancer naturally originating in a portion of a patient selected from the group consisting of: stomach, colon, rectum, mouth/pharynx, esophagus, larynx, liver, pancreas, lung, breast, cervix uteri, corpus uteri, ovary, prostate, testis, bladder, skin, bone, kidney, brain/central nervous system, head, neck, and throat.

16. A method of performing chemotherapy or radiation therapy on a patient, comprising:
(a) administering chemotherapy or radiation therapy to a patient diagnosed as needing chemotherapy or radiation therapy; and
(b) before, during, or after step (a), administering to the patient a therapeutically effective amount of a composition comprising carbon monoxide.

17. The method of claim 16, wherein the composition is administered before step (a).

18. The method of claim 16, wherein the composition is administered during step (a).

19. The method of claim 16, wherein the composition is administered after step (a).

20. The method of claim 16, wherein the composition is administered before, during, and after step (a).

21. The method of claim 16, wherein the composition is in gaseous form and is administered to the patient via inhalation.

22. The method of claim 16, wherein the composition is in gaseous form and is administered topically to an organ of the patient other than the patient's lungs.

23. The method of claim 16, wherein the composition is in liquid form and is administered the patient orally.

24. The method of claim 16, wherein the composition is in liquid form and is administered topically to an organ of the patient.

25. A method of performing surgery to remove naturally arising cancer from a patient, comprising:
(a) identifying in a patient at least one organ bearing naturally arising cancerous tissue;
(b) performing surgery on the patient to remove at least a part of the cancerous tissue; and
(c) before, during, or after step (b), administering to the patient a therapeutically effective amount of a composition comprising carbon monoxide.

26. The method of claim 25, wherein the composition is administered before step (b).

27. The method of claim 25, wherein the composition is administered during step (b).

28. The method of claim 25, wherein the composition is administered after step (b).

29. The method of claim 25, wherein the composition is administered before, during, and after step (b).

30. The method of claim 25, wherein the composition is in gaseous form and is administered to the patient via inhalation.

31. The method of claim 25, wherein the composition is in gaseous form and is administered topically to a site of the surgery.

32. The method of claim 25, wherein the composition is in liquid form and is administered the patient orally.

33. The method of claim 25, wherein the composition is in liquid form and is administered topically to the organ of the patient.

34. The method of claim 25, wherein the cancer is cancer naturally originating in a portion of a patient selected from the group consisting of: stomach, colon, rectum, mouth/pharynx, esophagus, larynx, liver, pancreas, lung, breast, cervix uteri, corpus uteri, ovary, prostate, testis, bladder, skin, bone, kidney, brain/central nervous system, head, neck, and throat.

35. A method of treating naturally arising cancer in a patient, comprising:
(a) identifying a patient suffering from or at risk for naturally arising cancer;
(b) providing a vessel containing a pressurized gas comprising carbon monoxide gas;
(c) releasing the pressurized gas from the vessel, to form an atmosphere comprising carbon monoxide gas; and
(d) exposing the patient to the atmosphere, wherein the amount of carbon monoxide in the atmosphere is sufficient to treat cancer in the patient.

36. The method of claim 35, wherein the patient is exposed to the atmosphere continuously for at least one hour.

37. The method of claim 35, wherein the patient is exposed to the atmosphere continuously for at least six hours.

38. The method of claim 35, wherein the patient is exposed to the atmosphere continuously for at least 24 hours.

39. The method of claim 35, wherein the patient is exposed to the atmosphere continuously for at least three days.

40. The method of claim 35, wherein the patient is exposed to the atmosphere continuously or intermittently over a period of at least one week.

41. The method of claim 35, wherein the patient is exposed to the atmosphere continuously or intermittently over a period of at least four weeks.

42. The method of claim 35, wherein the patient is exposed to the atmosphere continuously or intermittently over a period of at least one year.

43. The method of claim 35, wherein the cancer is cancer naturally originating in a portion of a patient selected from the group consisting of: stomach, colon, rectum, mouth/pharynx, esophagus, larynx, liver, pancreas, lung, breast, cervix uteri, corpus uteri, ovary, prostate, testis, bladder, skin, kidney, brain/central nervous system, head, neck, and throat.

44. The method of claim 35, wherein the concentration of carbon monoxide in the atmosphere is about 0.01% to about 0.22% by weight.

45. The method of claim 35, wherein the patient is a human.

46. A method of treating cancer in a patient, comprising:
administering to a patient diagnosed as suffering from or at risk for naturally arising cancer a therapeutically effective amount of a composition comprising carbon monoxide, wherein the patient is not a rodent.

47. A method of treating cancer in a human patient, comprising:
administering to a human patient diagnosed as suffering from or at risk for naturally arising cancer a therapeutically effective amount of a composition comprising carbon monoxide.

48. A method of treating cancer in a patient, comprising:
determining whether cancerous cells in a patient express p21; and
administering to the patient a therapeutically effective amount of a composition comprising carbon monoxide if the cancerous cells express p21.

49. A method of treating unwanted angiogenesis in a patient, comprising:
administering to a patient diagnosed as suffering from or at risk for unwanted angiogenesis a therapeutically effective amount of a composition comprising carbon monoxide.

50. The method of claim 49, wherein the composition is in gaseous form and is administered to the patient via inhalation.

51. The method of claim 49, wherein the composition in is gaseous form and is administered topically to an organ of the patient.

52. The method of claim 49, wherein the composition in is gaseous form and is administered to the abdominal cavity of the patient.

53. The method of claim 49, wherein the composition is in liquid form and is administered to the patient orally.

54. The method of claim 49, wherein the composition is in liquid form and is administered topically to an organ of the patient.

55. The method of claim 49, wherein the composition is in liquid form and is administered to the abdominal cavity of the patient.

56. A method of treating a condition associated with unwanted angiogenesis, comprising:
administering to a patient diagnosed as suffering from or at risk for a condition associated with unwanted angiogenesis a therapeutically effective amount of a composition comprising carbon monoxide, wherein the condition associated with unwanted angiogenesis is not cancer.

57. The method of claim 56, wherein the condition is selected from the group consisting of: rheumatoid arthritis, lupus, psoriasis, diabetic retinopathy, retinopathy of prematurity, macular degeneration, corneal graft rejection, neovascular glaucoma, retrolental fibroplasia, rubeosis, Osler-Weber Syndrome, myocardial angiogenesis, plaque neovascularization, telangiectasia, and angiofibroma.

58. A vessel comprising medical grade compressed carbon monoxide gas, the vessel bearing a label indicating that the gas can be used to treat cancer in a patient.

59. The vessel of claim 58, wherein the carbon monoxide gas is in admixture with an oxygen-containing gas.

60. The vessel of claim 58, wherein the carbon monoxide gas is present in the admixture at a concentration of at least about 0.025%.

61. The vessel of claim 58, wherein the carbon monoxide gas is present in the admixture at a concentration of at least about 0.05%.

62. The vessel of claim 58, wherein the carbon monoxide gas is present in the admixture at a concentration of at least about 0.10%.

63. The vessel of claim 58, wherein the carbon monoxide gas is present in the admixture at a concentration of at least about 1.0%.

64. The vessel of claim 58, wherein the carbon monoxide gas is present in the admixture at a concentration of at least about 2.0%.

65. A vessel comprising medical grade compressed carbon monoxide gas, the vessel bearing a label indicating that the gas can be used to prevent unwanted angiogenesis in a patient, or to treat a condition, other than cancer, associated with unwanted angiogenesis.

66. The vessel of claim 65, wherein the carbon monoxide gas is in admixture with an oxygen-containing gas.

67. The vessel of claim 65, wherein the carbon monoxide gas is present in the admixture at a concentration of at least about 0.025%.

68. The vessel of claim 65, wherein the carbon monoxide gas is present in the admixture at a concentration of at least about 0.05%.

69. The vessel of claim 65, wherein the carbon monoxide gas is present in the admixture at a concentration of at least about 0.10%.

70. The vessel of claim 65, wherein the carbon monoxide gas is present in the admixture at a concentration of at least about 1.0%.

71. The vessel of claim 65, wherein the carbon monoxide gas is present in the admixture at a concentration of at least about 2.0%.
